(19) United States
(12) Patent Application Publication
(10) Pub. No.: US 2014/0163394 A1
(43) Pub. Date: Jun. 12, 2014

(54) Neuropathy Test Device

(71) Applicant: Timothy Katz, Lewes (GB)

(72) Inventor: Timothy Katz, Lewes (GB)

(73) Assignee: University of Brighton, Brighton, East Sussex (GB)

(21) Appl. No.: 14/114,046

(22) PCT Filed: Apr. 26, 2012
(86) PCT No.: PCT/GB2012/050915

§ 371 Date: Dec. 16, 2013

(30) Foreign Application Priority Data
Apr. 26, 2011 (GB) 1107046.3

(51) Int. Cl. A61B 5/0205; A61B 5/00

(52) U.S. Cl. CPC A61B 5/0205; A61B 5/483
USPC 600/483; 600/555

(57) ABSTRACT

A device for testing for the presence of neuropathy by applying a thermal stimulus to a test area of skin includes a temperature sensing arrangement for determining a reference temperature being a reference skin temperature or room temperature, a thermal stimulus application arrangement and a temperature controlling arrangement for controlling temperature of the thermal stimulus. The reference temperature is used in controlling the temperature of the applied thermal stimulus. The device may include a blood flow sensing arrangement which provides a measurement of blood flow in an area of the skin which may be considered to be indicative of a blood flow in response to an applied stimulus.

The present invention relates to a device for testing for the presence of neuropathy. More specifically the invention is concerned with a device for testing for the presence of neuropathy by applying a thermal stimulus to a test area of skin, (a “thermal neuropathy test device”). The test device is particularly applicable to testing for peripheral neuropathy.

Peripheral neuropathy is a condition in which nerves of the peripheral nervous system become damaged. This may be as a result of systemic illness or diseases of the nervous system. One condition which is commonly associated with damage to the peripheral nervous system is diabetes. It is well known that diabetes is an increasing problem, thought to be as a result of modern lifestyle factors. Peripheral neuropathy is therefore also an increasingly common condition.

It is important to be able to identify the presence of peripheral neuropathy at an early stage in order to treat the condition and try to prevent its progression. Peripheral neuropathy is a serious and irreversible disease, which may ultimately be fatal without appropriate intervention. For example, neuropathy of nerves in the feet may mean that the affected patient is unable to feel when skin has become damaged, e.g. by rubbing. Individuals having peripheral neuropathy are also often more likely to suffer from skin damage due to an underlying condition e.g. diabetes. If the skin becomes damaged and is not treated, blisters may form which may progress to become ulcers. In some cases skin damage may become so severe that amputation of the affected limb is the only way to contain the damage. A leading reason for non traumatic amputations is damage as a consequence of diabetes.

Various tests have been proposed to try to identify when peripheral neuropathy exists in a patient. Some of these tests are thermal tests, involving the application of thermal stimulus to a test area of skin and determining the response of a patent thereto. For example, such tests are commonly applied to the feet.

One type of thermal test involves the use of a double ended probe, having one end that is metal and one that is plastic. The plastic and metal ends are held against the skin in turn to determine whether the patient is able to differentiate between the relatively cold and hot thermal stimuli provided by the metal and plastic ends. However, such a test has certain limitations. Depending upon the temperature of the surrounding environment, the temperature of the probe or the skin of the patient may vary. If the skin is relatively cold, for example, the metal end may not be significantly colder than the skin temperature, such that failure to notice a cold sensation might not reliably indicate an underlying problem. Thus the outcome of the test is highly dependent upon the ambient conditions, making it difficult to draw reliable conclusions as to whether peripheral neuropathy exists.

Another thermal test involves preparing test tubes filled respectively with hot and cold water, which are held against a test area of skin to stimulate a patient response. Such a method also suffers from certain drawbacks. For example, it is difficult to accurately control the temperature that is applied to the skin using this technique. Delays in preparing the test tubes and applying them to the skin may mean that the actual temperature applied may not correspond to the intended temperature. Furthermore operation of this type of test is inherently cumbersome and results susceptible to variation due to environmental conditions for the same reasons as the double ended probe test.

A further problem the Applicant has identified is that both of the types of thermal stimulus test described above provide inherently subjective results, and are incapable of giving a calibrated response, instead providing only an approximate “yes” or “no” type response. The Applicant has realised that there is a need for an improved thermal neuropathy test device.

In accordance with a first aspect of the invention there is provided,

a neuropathy test device comprising;

a thermal stimulus application arrangement for applying a thermal stimulus to a test area of skin in use,

wherein the neuropathy test device further comprises a temperature sensing arrangement for sensing a reference temperature, wherein the reference temperature is a temperature of the ambient environment or a reference skin temperature.

In accordance with the invention therefore, the neuropathy test device is arranged to apply a thermal stimulus to an area of skin (the “test area”). The device also includes a temperature sensing arrangement for sensing a reference temperature. The reference temperature may be used in controlling the temperature of an applied thermal stimulus. The thermal stimulus application arrangement and the temperature sensing arrangement are provided in a single test device.

As mentioned above, the Applicant has realised that a drawback of certain prior art neuropathy test devices is that no account is taken of the temperature of the ambient environment and/or the base temperature of the skin. Accordingly, results obtained may be significantly affected by environmental factors. For example, a thermal stimulus intended to provide a hot sensation may not in fact have a temperature significantly different to the skin temperature when ambient conditions are warm. A false positive result for neuropathy may be obtained if a patient is not able to detect such a stimulus. Similarly, a thermal stimulus involving a transition between two temperatures which should feel respectively hot and cold may not provide reliable results if the skin temperature is in fact above or below both temperatures.

In accordance with the invention, a temperature sensing arrangement is provided, which senses a reference temperature for use when carrying out testing of an area of the skin using the thermal stimulus application arrangement. The thermal stimulus application arrangement may apply a thermal stimulus to the test area of skin taking into account the reference temperature. This enables more accurate results to be obtained when testing for neuropathy using the device, allowing the ambient temperature e.g. of a room in which the test is performed, and/or a base skin temperature to be taken into account. The reference temperature may be used to set the temperature of an applied stimulus to ensure that it provides a relatively hot or cold sensation as intended with respect to a base temperature of the skin as indicated by the reference temperature. This enables more reliable conclusions regarding the presence of neuropathy to be derived. The temperature sensing arrangement is incorporated in the same device as the thermal stimulus application arrangement providing ease of use.

The reference temperature may be an ambient temperature i.e. a temperature of the ambient or external environment. In some embodiments it is a room temperature. The skin temperature of a patient may be assumed to vary in a particular manner with ambient temperature. Thus by measuring the ambient e.g. room temperature a reference temperature may be obtained which may be correlated to or taken as a reference skin temperature. However, preferably the reference temperature is a reference skin temperature, and the temperature sensing arrangement is arranged for sensing such a reference temperature. This enables more direct and accurate control of the temperature of a thermal stimulus relative to the skin temperature to be obtained.

In accordance with a second aspect of the invention there is provided a neuropathy test device comprising;

a thermal stimulus application arrangement for applying a thermal stimulus to a test area of skin in use, wherein the test device further comprises a temperature sensing arrangement for sensing a reference temperature, preferably wherein the reference temperature is a temperature of the ambient environment or a reference skin temperature. The reference temperature is for use in controlling the temperature of a thermal stimulus applied by the thermal stimulus application arrangement.

The present invention in this second aspect may include any or all of the features described in relation to the other aspects or embodiments of the invention and vice versa. It will be appreciated that the features described herebelow are applicable to the invention in accordance with the first or this further aspect, and the discussion above is applicable to the present invention in accordance with the first or the further aspect of the invention. For the avoidance of doubt, any of the features described herein in relation to a given aspect of the invention are equally applicable to any other aspect of the invention if not explicitly stated, unless mutually inconsistent.

In embodiments of the invention in any of its aspects or embodiments, the device is an electronic device.

In accordance with the invention in any of its aspects or embodiments, the device preferably further comprises a temperature controlling arrangement operable to control a temperature of the thermal stimulus applied by the thermal stimulus application arrangement taking into account a reference temperature sensed by the temperature sensing arrangement. The temperature controlling arrangement comprises a driver for a heat transfer arrangement of the thermal stimulus application arrangement. In embodiments the temperature controlling arrangement comprises temperature controlling circuitry operable to control a temperature of the thermal stimulus. In some preferred embodiments the device is arranged such that a sensed reference temperature e.g. a signal indicative thereof, is input to the temperature controlling arrangement for use in controlling the temperature of the applied thermal stimulus. This may be achieved using suitable circuitry. The sensed reference temperature that is input to the temperature controlling arrangement may be the output of the temperature sensing arrangement or the result of processing the output thereof.

The temperature controlling arrangement may be operable to control the temperature of the thermal stimulus taking into account a sensed reference temperature in any suitable manner. The temperature controlling arrangement is operable to control the temperature of an applied thermal stimulus using the sensed reference temperature. The reference temperature may be used directly or indirectly by the temperature controlling arrangement to control the temperature of the thermal stimulus. Preferably the temperature controlling arrangement is operable to control the temperature of the thermal stimulus relative to the reference temperature.

The temperature controlling arrangement is operable to control the temperature of the thermal stimulus applied taking into account at least the sensed reference temperature. In other words, the temperature controlling arrangement is operable to control the temperature of the thermal stimulus with reference to at least the sensed reference temperature. In some embodiments other parameters may also be used to control the temperature of the thermal stimulus. For example, the temperature of a thermal stimulus application arrangement applicator surface may be used to control the temperature of the thermal stimulus applied in a feedback arrangement. Other parameters may be specified by a user or may be predetermined, e.g. defining temperature settings for the thermal stimulus.

In preferred embodiments the temperature controlling arrangement is operable to control the temperature of an applied thermal stimulus in accordance with one or more temperature settings taking into account the sensed reference temperature. Thus the temperature controlling arrangement controls the temperature of the applied thermal stimulus in a manner so as to (try to) achieve a given temperature setting. In some preferred embodiments the temperature controlling arrangement is in communication with a temperature setting arrangement for providing one or more temperature settings for the thermal stimulus to be applied, and is arranged to control the temperature of the thermal stimulus taking into account the reference temperature in accordance with a given temperature setting provided by the temperature setting arrangement.

The temperature setting arrangement may act as a controller for the temperature controlling arrangement. The temperature setting arrangement may comprise a set of one or more processors. In embodiments in which the temperature controlling arrangement is in communication with a temperature setting arrangement, the device may comprise the temperature setting arrangement, i.e. the temperature setting arrangement may be an integral part of the device. In other embodiments, the temperature setting arrangement may be separate from the device and operably connected thereto. The connection may be via a wireless or wired communications link. For example the temperature setting arrangement may then be provided by a separate computing apparatus to which the neuropathy test device is connected or a set of one or more processors thereof. For example, the temperature setting arrangement may be provided by a program run on a processor. The present invention extends to a neuropathy test apparatus comprising the test device of the invention in any of its aspects and embodiments and the temperature setting arrangement.

In some embodiments the temperature setting arrangement is arranged to provide a control signal to the temperature controlling arrangement indicative of a temperature setting for a thermal stimulus to be applied. The control signal may provide an input to the temperature controlling arrangement. It will be appreciated that the sensed reference temperature may then additionally be input to the temperature controlling arrangement. In other arrangements a sensed reference temperature may be input to the temperature setting arrangement and used by the temperature setting arrangement to control the temperature controlling arrangement in accordance with a given temperature setting taking into account the sensed reference temperature. For example, a single control signal may be provided by the temperature setting arrangement to the temperature controlling arrangement.

In accordance with the invention in any of its embodiments in which the temperature controlling arrangement is operable to control the temperature of the thermal stimulus in accordance with a temperature setting, the temperature setting may be of any type. In accordance with the invention, the temperature controlling arrangement may be arranged such that the reference temperature is used to determine a base temperature level for use in controlling the temperature of the thermal stimulus in accordance with a temperature setting. The reference temperature may be used as the base temperature or the base temperature may be set relative to the reference temperature. In one preferred embodiment, the temperature controlling arrangement is operable to control the temperature of an applied stimulus in accordance with a temperature setting in which the temperature of the stimulus applied corresponds to a base temperature, and/or wherein the temperature of the stimulus is arranged to change from a base temperature, wherein the temperature controlling arrangement is arranged to use the reference temperature to determine the base temperature. In preferred embodiments the reference temperature is used as the base temperature. This may be appropriate where the reference temperature is a reference skin temperature. If the reference temperature is an ambient e.g. room temperature, a correction factor may need to be applied to correlate the temperature to a skin temperature.

A given temperature setting may comprise setting a temperature of the thermal stimulus to a base level, and/or to one or more given levels relative to a base level. Alternatively or additionally a temperature setting may involve a change in temperature, e.g. a transition between given temperature levels and/or a change in temperature level from a given level. In any of these types of setting, the given temperature level(s) may include a base temperature level. A transition may comprise a continual transition or a stepped transition. A stepped transition may include one or more discrete steps. In embodiments in which a temperature setting involves a change in temperature from a given level, or a transition between temperature levels, the temperature controlling arrangement may be arranged to change the temperature level at a given rate.

The present invention enables the temperature of the applied stimulus to be set in any desired manner which may be useful in testing for neuropathy based on e.g. relative to the sensed reference temperature. Some examples are discussed in the detailed description below. It will be appreciated that the references to the temperature of the thermal stimulus refer to a target temperature thereof, i.e. a temperature that it is desired to achieve.

The temperature settings are predetermined settings. The temperature settings may be preset settings e.g. factory set or may be user specified settings. Regardless of how the temperature settings are set, preferably the temperature settings are user selectable temperature settings. It is preferred that the settings may be customised by a user. In some preferred embodiments the device is programmable by a user to provide temperature settings. The device may comprise or may be connectable to a set of one or more processors for this purpose. The device may be directly programmable or may be connected to a computing device to enable programming of temperature settings. The invention extends to a neuropathy test apparatus comprising the device wherein the apparatus is configured such that the device is programmable. The device may then be connected to a set of one or more processors for this purpose.

In other embodiments the device may be arranged to automatically determine a temperature setting or settings taking into account the reference temperature. The device may be arranged to select a temperature setting or settings (e.g. relative to the reference temperature) pseudo randomly. In some embodiments the device may be arranged to pseudo randomly select a temperature setting that is above or below the reference temperature. The device may automatically select a temperature setting, e.g. pseudo randomly, between given limits, respectively above and below the reference temperature to provide a hot or cold stimulus. This may prevent the operator from giving any conscious or subconscious hint to the patient as to whether to expect a hot or cold stimulus, effectively providing a double blind test.

In accordance with the invention in any of its embodiments, the test area of skin is an area of skin of a patient being tested for neuropathy. A patient refers to any subject being tested for the presence of neuropathy. It will be appreciated that the invention may be applied to testing of human or animal subjects. The device is particularly applicable to testing for the presence of peripheral neuropathy. In embodiments in which the reference temperature is a reference skin temperature, the reference skin temperature is desirably a temperature of a skin surface including the area to be tested. The reference temperature may be, for example, a temperature of a region within the area to be tested or of a region in the vicinity of the area to be tested e.g. forming part of the same skin surface. Ideally a reference skin temperature is taken in respect of a part of the skin whose temperature may be assumed to correspond to that of the skin area to be tested. This may avoid the need to carry out any corrections to the reference temperature.

The device may be arranged such that the temperature sensing arrangement may be operated to sense a skin temperature before and/or at the same time as the thermal stimulus application arrangement is operated to apply a thermal stimulus to a skin test area. Preferably the temperature sensing arrangement is operable to sense a reference, e.g. skin temperature at least before the thermal stimulus application arrangement is operated.

In embodiments in which the temperature sensing arrangement may be operated at the same time as the thermal stimulus application arrangement, a reference skin temperature may be sensed e.g. continually or intermittently during delivery of a thermal stimulus. This may provide a way of verifying that an intended thermal stimulus is being delivered. The temperature controlling arrangement may control the temperature of the applied thermal stimulus accordingly based upon the detected reference temperature. This may provide a way of controlling the thermal stimulus application arrangement more accurately to try to achieve a desired temperature setting in a feedback type arrangement. In many situations it may be assumed that the temperature of the skin and the temperature of an applicator surface of the thermal stimulus application arrangement correspond to one another or may at least be correlated to one another. Thus by sensing the temperature of the skin during application of a thermal stimulus, the temperature of the thermal stimulus being delivered may be inferred, and used to verify that the intended stimulus is being applied, and if desired, to control the temperature applied in a feedback arrangement. This may be used alternatively or additionally to sensing the temperature of a part of the thermal stimulus arrangement. Sensing of a part of the stimulus application arrangement e.g. an applicator surface is preferred as it may provide a more accurate indication of an applied temperature even if thermal contact between the applicator surface and skin surface is not good or is interrupted.

The temperature sensing arrangement for sensing a reference temperature may comprise one or more temperature sensors. The temperature sensing arrangement may comprise a contact temperature sensor, or more preferably a non contact temperature sensor. The temperature sensing arrangement may include only a non contact temperature sensor or sensors. Most preferably the temperature sensing arrangement comprises an infrared temperature sensor. Non contact i.e. remote temperature sensors are preferred, as they may be used to determine a skin reference temperature without stimulating the nerves or influencing the temperature of the skin, thus providing a more accurate result, and reducing the likelihood of any perceived change in the skin temperature as a result of the temperature sensing process.

The temperature sensing arrangement for sensing a reference temperature is arranged to sense a reference temperature and provide an output e.g. output signal which is indicative of the sensed reference temperature. The output is the result of the sensing i.e. the interaction of the sensor with the skin or ambient environment. In other words, the temperature sensing arrangement is arranged to provide an output dependent upon a temperature sensed. As described above, the temperature sensing arrangement may interact with the ambient environment or skin to provide the output indicative of the sensed reference temperature. The sensing arrangement interacts with the environment external to the device to provide the reference temperature, and the sensing arrangement is configured to determine the temperature of skin or the environment external to the device.

The output indicative of the sensed reference temperature may be in the form of an electrical signal or similar which is dependent upon temperature. As described above, the output may be used directly or indirectly to control the operation of the thermal stimulus application arrangement. The output of the temperature sensing arrangement may be provided directly to e.g. a temperature controlling arrangement for use in controlling the temperature of the applied thermal stimulus. In other embodiments it may be subjected to some processing first. Processing may be used, for example, to correct for any sensor bias, convert an analogue output to a digital output, or to convert the output of the sensing arrangement to a form that may is intelligible to a user of the device. This may be appropriate if it is desired for the device to be able to provide a display of the reference temperature to a user.

In some embodiments the temperature sensing arrangement may comprise or be in communication with a set of one or more processors for processing an output of the temperature sensing arrangement and/or controlling operation of the temperature sensing arrangement. The set of one or more processors may be integral with the device, or may be separate from the device and in communication therewith. In some embodiments a temperature sensor of the sensing arrangement may incorporate a set of one or more processors. The invention extends to a neuropathy test apparatus comprising the device in accordance with any of its aspects or embodiments and a set of one or more processors in communication with the temperature sensing arrangement for processing the output of the temperature sensing arrangement and/or controlling operation of the temperature sensing arrangement.

In some preferred embodiments the temperature sensing arrangement for sensing a reference temperature, or at least a temperature sensor thereof is adjacent or in proximity to the thermal stimulus application arrangement, or at least a thermal stimulus applicator surface thereof. In some embodiments therefore a temperature sensor of the temperature sensing arrangement is adjacent to or in proximity to a thermal stimulus applicator surface of the thermal stimulus applicator arrangement. In some embodiments the device comprises a single face which includes the temperature sensing arrangement or at least a temperature sensor thereof and the thermal stimulus arrangement or at least an applicator surface thereof. The face faces the skin in use.

In accordance with any of the aspects or embodiments of the invention, the thermal stimulus application arrangement may be operable to apply a hot and/or cold stimulus to the test area, and most preferably is arranged to be selectively operable to apply either a hot stimulus or a cold stimulus to the test area. It will be appreciated that the thermal stimulus application arrangement is then operable to apply one of the hot stimulus or cold stimulus to the test area at a given time. As mentioned above, the thermal stimulus application arrangement may be operable to provide different stimuli selected from hot or cold stimuli, and/or to transition between different types of stimuli. The device may do this automatically and may automatically e.g. pseudo randomly apply a hot or cold stimulus, e.g. select a setting between given temperature limits above and below the reference temperature. The device may select between preset settings which may or may not be user set.

As used herein, a “hot stimulus” refers to a stimulus having a temperature above a base temperature of the skin, and a “cold stimulus” to one having a temperature below the base skin temperature, such that they should respectively result in a hot or a cold sensation being experienced by a patient. In embodiments in which the reference temperature is a reference skin temperature, the reference skin temperature will correspond to the base temperature of the skin, and thus hot and cold stimuli will be stimuli at temperatures respectively above and below the reference skin temperature. In embodiments the temperature of the thermal stimulus applied is controllable by the thermal stimulus temperature controlling arrangement.

The thermal stimulus application arrangement may be operable to apply a thermal stimulus to a test area of skin in the form of a single continuous area or one or more discrete sub areas. Most preferably the thermal stimulus application arrangement is operable to apply the thermal stimulus to a single continuous area of the skin. Preferably the thermal stimulus application arrangement is operable to apply the thermal stimulus to a test area of the skin of a foot. However, it will be appreciated that the thermal stimulus application arrangement may be operable to apply a thermal stimulus to a test area of the skin of any part of the body useful in testing for neuropathy. Regardless of the part of the body to which the test area belongs, the test area may be a localised area e.g. a point, or an extended area, such as the entire surface of the skin of the sole of a foot. The temperature sensing arrangement is preferably arranged for sensing the temperature of an area of skin of a corresponding part of the body to provide a reference skin temperature. By way of example, the test area may be in the range of from 5 cm² up to the area of the sole of a foot. In some embodiments the test area is an area of at least 5 cm². In some embodiments the test area is less than 500 cm².

In embodiments the thermal stimulus application arrangement comprises a heat transfer arrangement for delivering heat to and/or receiving heat from the test area of skin. The heat transfer arrangement may be operable to only deliver heat to the test area of skin for delivering a heat stimulus, or only to remove heat from the test area of skin for delivering a cold stimulus, but more preferably is selectively operable to deliver heat to the test area for delivering a heat stimulus or to remove heat from the test area of skin for delivering a cold stimulus. Thus the heat transfer arrangement is preferably bi-directional. The thermal stimulus temperature controlling arrangement may be operable to drive the heat transfer arrangement for controlling the temperature of an applied thermal stimulus. The temperature controlling arrangement may, for example, comprise an arrangement to control the direction of current flow through the heat transfer arrangement of the thermal stimulus application arrangement, to cause a relatively hotter or colder thermal stimulus to be applied. In some embodiments the temperature controlling arrangement comprises an H-bridge.

Any form of heat transfer arrangement may be used. For example, the heat transfer arrangement may be a chemical, electrical or mechanical heat transfer arrangement or any combination thereof. The heat transfer arrangement may comprise a heat pump, preferably a bi-directional heat pump. In some embodiments it is envisaged that the heat transfer arrangement may comprise a volatile vapour heat exchanger. However, preferably the heat transfer arrangement comprises a thermoelectric heat transfer device. Such devices may provide a rapid but controlled rate of change of temperature of the thermal stimulus. The thermoelectric heat transfer device may be a solid state heat transfer device. Most preferably the thermal stimulus arrangement comprises a Peltier device, such as a multilayer Peltier device. A multilayer Peltier device may include numerous silicon junctions.

In embodiments the thermal stimulus application arrangement comprises a thermal stimulus applicator surface in thermal communication with the heat transfer arrangement for contacting the test area of skin to apply the thermal stimulus thereto. The heat transfer arrangement will then be operable to deliver heat to or remove heat from the thermal stimulus applicator surface to thereby deliver a thermal stimulus to the test area of skin when the thermal stimulus applicator surface is in thermal contact therewith. The thermal stimulus applicator surface of the thermal stimulus arrangement may or may not directly contact the heat transfer arrangement of the thermal stimulus arrangement. Any suitable arrangement may be used provided that there is thermal communication between the thermal stimulus applicator surface of the thermal stimulus application arrangement and the heat transfer arrangement thereof. In some embodiments the thermal stimulus applicator surface may be a surface of the heat transfer arrangement. For example, the surface may be the surface of a Peltier pad. It will be appreciated that the thermal stimulus application arrangement may comprise one or more applicator surfaces for contacting the test area of skin. In some embodiments a single applicator surface is provided.

In some preferred embodiments the thermal stimulus application arrangement comprises an applicator plate defining the or a thermal stimulus applicator surface or surfaces. For example an applicator plate may be in the form of a foot plate upon which a test subject may stand in order for a thermal stimulus to be delivered to a test area of a sole of a foot. In other arrangements the applicator surface may be of a more limited area for applying a thermal stimulus to a localised area of the skin. In some embodiments the thermal stimulus application arrangement comprises an applicator pad defining the thermal stimulus applicator surface or surfaces.

The overall area of the applicator surface or surfaces may be selected as desired depending upon the size of the area of skin to be tested. The applicator surface or surfaces may have an area in any of the ranges described above for the test area. Where multiple surfaces are provided the total area of the surfaces may be in these ranges.

In embodiments the apparatus further comprises a temperature sensing arrangement for sensing the temperature of an applied thermal stimulus. The temperature sensing means may determine the temperature of any part of the thermal stimulus application arrangement which may be taken as representative of the temperature being applied to the test surface of skin. The temperature sensing arrangement may enable control of the temperature of the thermal stimulus to facilitate delivery of a thermal stimulus of a target temperature to the test skin surface. Preferably the temperature sensing arrangement is arranged to sense the temperature of a thermal stimulus applicator surface of the thermal stimulus application arrangement. The temperature sensing means may be of any suitable form. The temperature sensing arrangement for sensing the temperature of the thermal stimulus is preferably different to the temperature sensing arrangement for sensing the reference temperature. The temperature sensing arrangements may be of different types. In embodiments the temperature sensing arrangements for sensing the reference temperature and the temperature of the thermal stimulus are separate from one another i.e. distinct. In some embodiments the temperature sensing arrangement comprises a contact temperature sensor. In some embodiments the temperature sensing arrangement comprises a thermocouple.

The temperature sensing arrangement for sensing the temperature of the applied thermal stimulus interacts with a part of the thermal stimulus application arrangement to provide an output indicative of the temperature thereof. As described in relation to the temperature sensing arrangement for sensing a reference temperature, the output may be in the form of a signal which may be used to control the temperature controlling arrangement directly or indirectly. The output of the temperature sensing arrangement may be subjected to processing before being used to control the temperature controlling arrangement. In some embodiments the temperature sensing arrangement is in communication with a set of one or more processors for processing the output thereof and/or controlling the operation of the temperature sensing arrangement. The set of one or more processors may be arranged to use the output of the temperature sensing arrangement in controlling the temperature controlling arrangement. The processor or processors may be the same processors used to control the temperature controlling arrangement taking into account the sensed reference temperature and/or in accordance with a temperature setting described above. The set of one or more processors may or may not form an integral part of the device. In some arrangements the temperature sensing arrangement may itself comprise the set of one or more processors.

In embodiments in which a temperature controlling arrangement is provided for controlling the temperature of the thermal stimulus applied by the thermal stimulus applicator arrangement, the device may comprise a feedback arrangement arranged to use a sensed temperature of an applied thermal stimulus to control the temperature controlling arrangement in order to try to obtain a target thermal stimulus temperature. Such an arrangement may be implemented in any suitable manner. In some embodiments a sensed temperature of the thermal stimulus applied is fed back to an input of the temperature controlling arrangement for use in controlling the temperature of the applied stimulus.

In embodiments the thermal stimulus application arrangement comprises a heat sink in thermal communication with the heat transfer arrangement. The patient response to the thermal stimulus may be determined in any suitable manner. For example, the patient may be asked to indicate when they are able to detect the thermal stimulus. One commonly used test involves increasing or decreasing the temperature of an applied stimulus from a base level at a given rate and asking a patient to indicate when they are able to feel a cold or hot sensation. The time until the patient is able to feel a sensation is indicative of whether neuropathy is present. However, such methods involving eliciting patient responses are inherently subjective. The Applicant has realised that it would advantageous for results to be measured in a more objective manner.

In accordance with some preferred embodiments the device further comprises a blood flow sensing arrangement for sensing blood flow in a test area of skin to which a thermal stimulus is applied. In this way, the blood flow in an area of skin to which a thermal stimulus is being or has been applied may be sensed providing an objective assessment of the effect of the thermal stimulus on the patient. The blood flow in the area of skin responsive to an applied stimulus may be sensed. The blood flow rate may be sensed before, after and/or during application of a thermal stimulus to determine the effect of the stimulus. The blood flow rate sensing arrangement may or may not therefore be operable to sense the blood flow rate at the same time as the thermal stimulus is applied. A change in blood flow rate responsive to a thermal stimulus is an autonomous neural thermal response. In other words, it is a response which is involuntary, and out of the control of the patient. Sensing of blood flow may therefore provide an objective measure of the response to the thermal stimulus.

In accordance with a third aspect of the invention there is provided a neuropathy test device comprising;

a thermal stimulus application arrangement for applying a thermal stimulus to a test area of skin, and a blood flow sensing arrangement for sensing blood flow in the skin.

The present invention in this further aspect may include any or all of the features described in relation to the earlier aspects of the invention in any of their embodiments.

In those aspects and embodiments of the invention in which a blood flow sensing arrangement is provided, any form of blood flow sensor or sensors may be used. The blood flow sensing arrangement may be an optical blood flow sensing arrangement. In some preferred embodiments the blood flow sensing arrangement comprises a transmitter for transmitting electromagnetic radiation and a detector for receiving electromagnetic radiation. The transmitter and the detector may be separate or may be combined in a transceiver. The transmitter is operable to transmit electromagnetic radiation so as to be incident upon the surface of an area of skin to be tested and the detector for detecting electromagnetic radiation emanating from the skin surface. The amount of electromagnetic radiation absorbed may be used to make inferences regarding the blood flow in the area of skin upon which the electromagnetic radiation was incident as is well known in the art. By way of example, the sensing arrangement may be similar to those used to measure pulse rate using a fingertip mounted device. A change in blood flow and the implicit oxygenators will result in a change the absorption of electromagnetic radiation by the blood. The electromagnetic radiation is preferably infrared radiation. In preferred embodiments in which the temperature sensing arrangement for sensing a reference temperature comprises an infrared detector, the same detector may form the detector of the blood flow sensing arrangement. The device will then differ from an embodiment not having the blood flow sensing arrangement in that an infrared transmitter is additionally provided.

The blood flow sensing arrangement interacts with the skin to provide an output e.g. an output signal indicative of the blood flow in the skin. The output may be in the form of an electrical signal. In some arrangements, particularly where it is desired to provide a blood flow measurement result in a form which is intelligible to a user, e.g. via a display, it may be desirable to carry out some processing of the output of the sensing arrangement. The data may be processed to determine results regarding the presence or level of neuropathy. In embodiments in which the device comprises a blood flow sensing arrangement, a set of one or more processors is preferably provided in communication with the blood flow sensing arrangement for processing the output of the blood flow sensing arrangement and/or controlling operation thereof and/or for communication the results of the blood flow sensing. The set of one or more processors may be an integral part of the device and/or may be provided by a separate computing apparatus connected thereto. The invention extends to a neuropathy test apparatus comprising the device in accordance with any of the aspects of the invention and a set of one or more processors in communication with the blood flow sensing arrangement.

In some preferred embodiments the blood flow sensing arrangement, or at least a sensor thereof is adjacent or in proximity to the thermal stimulus application arrangement, or at least a thermal stimulus applicator surface thereof. In some embodiments therefore a sensor of the blood flow sensing arrangement is adjacent to or in proximity to a thermal stimulus applicator surface of the thermal stimulus applicator arrangement. In some embodiments the device comprises a single face which includes the blood flow sensing arrangement or at least a sensor thereof and the thermal stimulus arrangement or at least an applicator surface thereof. The face faces the skin in use.

Preferably the device in accordance with the invention in any of its aspects or embodiments comprises a display. The display may be used to display the temperature settings for the thermal stimulus, and/or to enable the user to interact with the device and/or to display results.

Preferably the device in accordance with any of the aspects and embodiments comprises a user interface. The user interface may allow the user to operate the sensing arrangements, initiate thermal stimulus application, select temperature settings or, where appropriate, program the device to customise settings. The user interface may also enable the user to input results, e.g. times until a patient responds to a stimulus, or may display results of tests in cases in which the device is capable of determining results e.g. through measurement of blood flow. The user interface may be of any type. In some embodiments the user interface comprises a touchscreen, a dial or dials, or a set of one or more buttons etc.

The device is a user operable device. The device may be manually operable.

In embodiments having a display or user interface, the operation of the display or user interface may be controlled by a set of one or more processors in communication therewith. The processor(s) may form a part of the device or may be provided as part of a separate apparatus connected thereto.

It will be appreciated that control of the device or any of its components to perform any of the functions described may be achieved, where not already described, under the control of a set of one or more processors. The present invention extends to a device of the invention in any of its aspects or embodiments, wherein the device is in communication with a set of one or more processors. The processors may or may not form part of the device. The one or more processors may be arranged for controlling the operation of the device or any of its components and/or processing an output of the device or any of its components. The present invention extends to a device in accordance with any of the aspects or embodiments of the invention wherein the reference temperature sensing arrangement and/or the blood flow sensing arrangement is in communication with a set of one or more processors for controlling operation thereof and/or processing an output thereof and/or communicating the results of the sensing.

The device may comprise a wired or wireless communications interface. The communications interface may enable the device to be placed in wired or wireless communication with a communications system. This may place the device in communication with a communications system for controlling operation of the device i.e. any of its functions and/or processing the output of one or more of the sensing arrangements, and/or for communicating the results of the neuropathy testing. The communications system may comprise a set of one or more processors.

Any reference to a processor may encompass a set of one or more processors.

Any connection or communication between components of the device or between the device and other apparatus may be via any suitable communications link, e.g. a wireless or wired communications link.

In accordance with any of the aspects and embodiments of the invention, the device may further comprise a power source. For example the power source may be a set of one or more batteries. In other arrangements, the device may be connectable to a power supply, e.g. via a suitable connector.

Preferably the device in accordance with the invention in any of its aspects or embodiments is a portable, and most preferably a hand held device. The device may then be of any suitable shape. In some embodiments the device may be in a shape resembling an electric razor.

The device may further comprise a housing. Any or all of the features described may be an integral part of the device which includes the temperature sensing arrangement and the thermal stimulus application arrangement, or the blood flow sensing arrangement and the thermal stimulus application arrangement in accordance with the different aspects of the invention. In embodiments comprising a set of one or more processors, the or each processor may or may not be provided as part of a separate apparatus. The invention will then extend to a neuropathy test apparatus comprising the device and the set of one or more processors in communication therewith.

In some preferred embodiments the device is a self contained device. In other words, all components, including e.g. any processors for controlling operation of the device form an integral part of the device, such that the device is provided by a single unit.

The present invention further extends to the use of a device in accordance with the invention in any of its aspects or embodiments to test for peripheral neuropathy.

The invention extends in a further aspect to a method of operating a device in accordance with the first or second aspect of the invention in any of its embodiments comprising the steps of operating the temperature sensing arrangement to obtain a reference temperature, and operating the thermal stimulus application arrangement to apply a thermal stimulus to the test area of skin taking into account the reference temperature. The method may further comprise operating a blood flow sensing arrangement where provided to determine the effect of the thermal stimulus upon blood flow e.g. in the test area.

The invention extends in a further aspect to a method of operating a device in accordance with the third aspect of the invention in any of its embodiments comprising the steps of operating the thermal stimulus application arrangement to apply a thermal stimulus to the test area of skin, and operating the blood flow sensing arrangement to determine the effect of the thermal stimulus upon blood flow e.g. in the test area. The method may further comprise operating the temperature sensing arrangement where provided to obtain a reference temperature for use in applying the thermal stimulus.

The present invention in these further aspects may include any or all of the features described in respect of the other aspects or embodiments of the invention.

References to “a processor” herein may refer to a set or one or more processors for carrying out the functions described. The same or different processors may be used to carry out any function or functions in relation to the device.

Some preferred embodiments of the present invention will now be described by way of example only and by reference to the accompanying drawings of which:

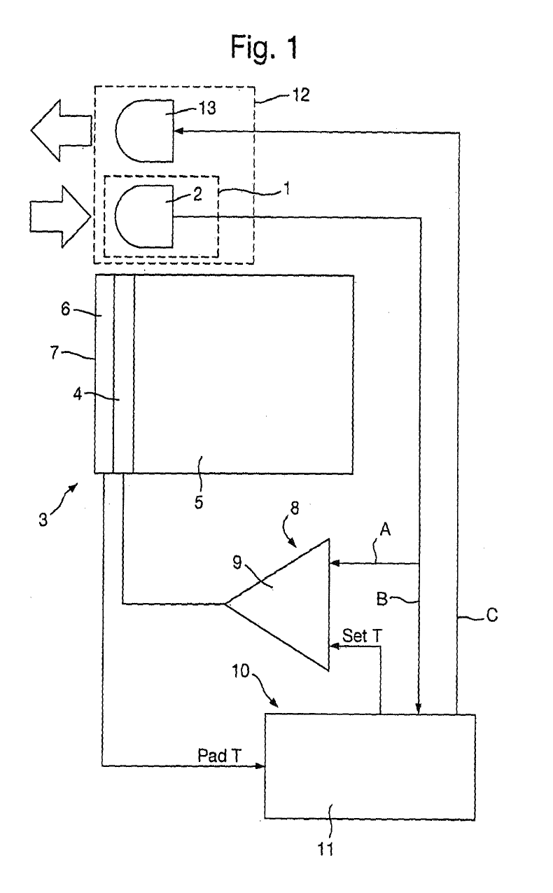

FIG. 1 is a diagram which schematically illustrates the circuitry of a neuropathy test device in accordance with a first embodiment of the invention;

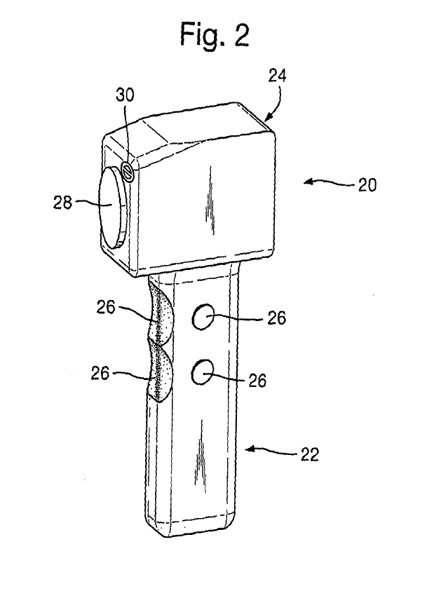

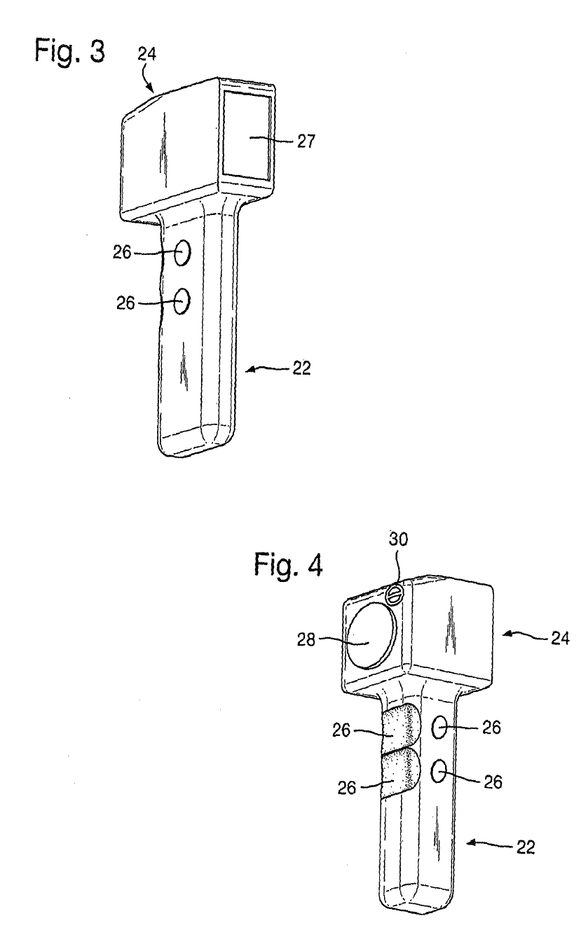

FIGS. 2, 3 and 4 are side and first and second perspective views respectively illustrating an exemplary device in accordance with one embodiment of the invention. FIGS. 3 and 4 are taken from a display screen and thermal stimulus application side of the device respectively.

Referring to FIG. 1, the neuropathy test device is an electronic device. The device includes a temperature sensing arrangement 1 for determining a reference temperature. In the illustrated embodiment the temperature sensing arrangement 1 includes an infrared detector 2. It will be appreciated that other forms of temperature sensing arrangement may be used for detecting the reference temperature. It is desirable that a remote i.e. non-contact temperature sensing arrangement is used to avoid influencing the skin temperature when a reference skin temperature is measured. The temperature sensing arrangement 1 is desirably used to determine a reference temperature that is a reference skin temperature. However the arrangement could also be used to determine a reference temperature that is a room temperature.

The device also includes a thermal stimulus application arrangement 3. In the illustrated embodiment the thermal stimulus application arrangement includes a bi-directional heat transfer arrangement, in the form of a thermoelectric device. The heat transfer device may be a bidirectional heat pump. In the exemplary arrangement the thermoelectric device is a Peltier device 4. The Peltier device 4 may be a multilayer device. The Peltier device 4 is in thermal communication with a heat sink 5. The thermal stimulus application arrangement further includes a skin contact pad 6 defining an applicator surface 7 for contacting an area of skin to apply the thermal stimulus thereto. The skin contact pad 6 is in thermal communication with the Peltier device 4 for delivering heat to or removing heat from the pad 6 and thus the applicator surface 7. In this way, when the surface 7 is placed in thermal contact with a test area of skin, a thermal stimulus will be applied thereto.

A temperature controlling arrangement 8 is provided for controlling the temperature of the thermal stimulus provided by the thermal stimulus application arrangement 3. In the illustrated embodiment the temperature controlling arrangement 8 includes a driver 9, which is operable to drive the Peltier device 4 so as to cause it to deliver heat to or remove heat from the contact pad 6, and thus the applicator surface 7, from or to the heat sink 5. The driver 9 may include an H-bridge.

A temperature setting arrangement 10 is in communication with the temperature controlling arrangement. In the device shown in FIG. 1, the temperature setting arrangement comprises a processor 11 which is arranged to provide a temperature setting function. The processor 11 provides an input labelled “Set T” to the driver 9. The input is a control signal causing the driver 9 to implement the intended temperature setting. In the embodiment illustrated, the processor 11 forms part of the device. However, it will be appreciated that in some embodiments rather than forming part of the device the processor may be provided as part of a separate apparatus to which the device may be connected via a wireless or wired communications link to provide to bring the processor into communication with the relevant parts of the device circuitry as described below. For example the processor could be a processor of a separate computing apparatus or similar. The temperature setting arrangement is operable to provide one or more temperature settings for the temperature controlling arrangement 8. The settings may be user selectable. For example, the device may include a user interface enabling a user to select a desired setting. Alternatively the device may be operable to pseudo randomly select a temperature setting. The device may pseudo randomly select whether a hot or cold stimulus is to be applied. The device may pseudo randomly select temperature settings between given limits above and below the reference temperature. This may provide a “double blind” type test, in which the test administrator does not know whether the stimulus being applied is hot or cold. Temperature settings may be preset. However, desirably the temperature setting arrangement is additionally or alternatively programmable to enable a user to customise temperature settings.

A temperature sensing arrangement (not shown) may be provided to sense the temperature of the applicator surface 7 of the pad 3 in order to measure the temperature of the thermal stimulus actually being applied. This may be achieved using a thermocouple for example or some other form of contact temperature sensor in thermal contact with the pad. The output of the temperature sensing arrangement may be provided to the temperature controlling arrangement 8 directly or via the processor 11 in a feedback type arrangement to try to deliver a target temperature. In FIG. 1 the output is shown as being provided to processor 11 for controlling the driver 9 along the line labelled “pad T”.

The device of FIG. 1 additionally includes a blood flow sensing arrangement 12. This enables a blood flow rate in the skin to be measured in order to provide a more objective measurement of the effect of an applied stimulus. The blood flow sensing arrangement provides a measurement of blood flow in an area of the skin which may be considered to be indicative of a blood flow in response to an applied stimulus. Thus the measurement area is close to the test area, or may incorporate the test area, provided that it is sufficiently in proximity to the test area to be representative of a change responsive to the stimulus. In the embodiment illustrated the blood flow measurement arrangement includes an electromagnetic radiation emitter and detector pair i.e. an infrared transceiver. The electromagnetic radiation emitter is an infrared emitter 13, and the detector is the infrared detector 2 which additionally measures the reference temperature. The operation of the blood flow measurement arrangement may be controlled by the processor 11. The processor 11 may process an output signal obtained from the infrared emitter and/or detector to obtain blood flow results which may be provided to a user. It will be appreciated that rather than utilizing the same infrared detector 1 for determining both a reference temperature and also as part of a blood flow sensing arrangement, an entirely separate blood flow measurement arrangement may be provided e.g. by providing a further infrared detector. Techniques for determining blood flow using an infrared detector and receiver arrangement are known, and involve consideration of the amount of radiation received from the surface after radiation is directed at the surface of the skin. For example such techniques are described in more detail in US 2008/0076984 A1.

It will be appreciated that while in preferred embodiments the blood flow sensing arrangement is incorporated in the device, this is only optional. A device without this capability would be constructed without the additional infrared emitter 13.

In FIG. 1 the connection between the components of the device is schematically illustrated. The temperature sensing arrangement 1 is connected to the temperature controlling arrangement 8 for providing a sensed reference temperature to the temperature setting arrangement for use with the temperature setting signal provided by the temperature setting arrangement 10 to control the temperature of the thermal stimulus provided by the thermal stimulus application arrangement 3. This connection between the infrared detector 2 and the driver 9 in the embodiment shown is along the line labelled “A”. The sensed reference temperature provides an input to the driver 9. The temperature sensing arrangement 1 may be arranged to carry out some processing of the sensing results to provide the output for communication to the driver 9. It will be appreciated that the output of the temperature setting arrangement 1 may alternatively or additionally be provided to the temperature setting arrangement 10 or processor 11 for processing before being used to control the temperature controlling arrangement 8 taking into account the sensed temperature. Thus in addition or alternatively to being provided to the driver 9 along line A, the output of the temperature sensing arrangement 1 may be provided to the processor 11 along line B.

The driver 9 has a second input connected to the processor 11, labelled “Set T” as described above for providing a temperature setting to the driver. The temperature controlling arrangement 8 is connected to the thermal stimulus application arrangement 3 for controlling the temperature thereof. In the illustrated embodiment, the output of the driver 9 is connected to an input of the Peltier device 4. The output of a temperature sensing arrangement for sensing the temperature of the applicator surface 7 may additionally be fed back to the driver 9 to improve control of the applied temperature. In FIG. 1 this is shown by the line labelled “pad T” which illustrates the output being provided to the processor 11 for use in controlling the driver 9. In the illustrated embodiment which includes the blood flow sensing arrangement 12, the infrared detector 13 provides an input to the processor along the line “B” to enable the processor 11 to derive blood flow results. The processor 11 is then also connected to the emitter of the blood flow sensing arrangement 12 along the line C for controlling operation of the sensor arrangement.

In accordance alternative embodiments in accordance with the second aspect of the invention, it is envisaged that the reference temperature sensing means 1 may be omitted. Thus such an arrangement would be as shown in FIG. 1, except that the infrared detector 2 would not be used to determine a reference temperature, and there would be no input to the driver 9 along line A. The infrared detector 2 would still act with the infrared emitter 13 to provide a blood flow sensing arrangement 12.

Operation of the device shown in FIG. 1 will now be described.

A reference temperature of the skin is first determined. This is achieved using the infrared detector 2 of the reference temperature sensing arrangement 1. It will be appreciated that the part of the skin whose temperature is measured need not correspond exactly to the area which is to be subjected to a thermal stimulus, provided that its temperature may be taken as being representative of the area which is to be tested. In the embodiment shown, the temperature of an area adjacent the test area will be measured. In accordance with other embodiments, rather than obtaining a reference skin temperature, the temperature sensing arrangement 1 may be used to instead determine a reference temperature which is a temperature of a room in which the test is being performed i.e. an ambient temperature.

The detected reference temperature is input to the driver 9 of temperature controlling arrangement 8 along line A. The device is located such that the applicator surface 7 of the applicator pad 6 contacts the skin over an area to which it is desired to deliver a thermal stimulus.

The temperature setting arrangement 10 is used provide a temperature setting which is also input to the driver in the form of a control signal along line Set T. This may be a preset temperature setting, or may be a user selected setting.

Once the user initiates a thermal stimulus application e.g. using an appropriate control switch, the driver 9 drives the heat transfer arrangement of the thermal stimulus application arrangement 3 i.e. the Peltier device 4 to deliver heat to or remove heat from the applicator surface 7 of the applicator pad 6 in accordance with the provided temperature setting and taking into account the reference temperature. In this way a thermal stimulus is applied to the skin. During application of the stimulus, the temperature of the applicator surface 7 is sensed, and fed back to the processor 11 along line “pad T” to provide more accurate control of the applied temperature.

A response of the user to the applied stimulus may be assessed using conventional techniques. For example, the user may be asked to indicate when they can feel an applied stimulus, and the time until a stimulus is sensed may be recorded to allow inferences regarding the presence of neuropathy to be determined. Alternatively, if provided, the blood flow sensing arrangement 12 may be operated to measure a change in blood flow in the skin responsive to the applied stimulus. This may provide more objective results, as blood flow is an autonomous response, i.e. one which is not under the control of the user. The infrared transmitter 13 may be operated to cause electromagnetic radiation to be incident on the skin, and the infrared detector 2 may detect radiation emanating from the surface. The amount of electromagnetic radiation absorbed by the skin as indicated by the input to the detector 2 may be used to determine a blood flow measurement. This may be achieved by providing a signal indicative of the received radiation to the processor 11 along line B.

The temperature sensing arrangement 1 may be operated to sense a reference skin temperature during application of a thermal stimulus instead or in addition to before delivery of a stimulus to provide further control of the applied stimulus temperature. In this case the sensing of the temperature of the pad surface 7 may be omitted as the skin temperature may be taken to correlate or correspond to the pad temperature.

Some possible temperature settings will now be described, and the manner in which the reference temperature used discussed in more detail.

Any temperature setting useful in testing for neuropathy may be used. One temperature setting may involve a thermal stimulus which is initially at a base temperature, before increasing or decreasing from the base temperature. The rate of change of temperature may be controlled. It may then be determined at which point a user is able to sense the temperature, or, where blood flow sensing is used, a particular change in blood flow is detected. In some embodiments, the user may be asked to give a verbal indication or a physical indication such as pressing a button. The time until the indication or change is noted may be recorded. A timer may be coordinated with the stimulus. If the rate of change of temperature is known, the temperature at which a response is obtained may be derived if the temperature is not provided directly by the device.

Numerous other temperature settings may be envisaged. For example a single temperature level above or below a base temperature level may be applied, or a transition which may be stepped or gradual may be provided between two different temperature levels, which may or may not include a base temperature.

In accordance with the invention, the temperature setting is controlled to take into account the reference temperature. For example, a reference skin temperature may advantageously be taken to provide the base temperature for the temperature settings, or the base temperature may be chosen to have a defined relationship with respect to the reference temperature e.g. 4 degrees above or below etc. Thus a temperature of an applied stimulus may be controlled relative to a reference skin temperature. This may ensure that an initial or base temperature corresponds to the original temperature of the skin before the stimulus is applied, such that any change from this temperature does constitute a thermal stimulus as intended. A similar method may be used if a reference room temperature is taken, provided this may be assumed to correspond to the skin temperature, or a suitable correction factor may be applied to correlate the reference temperature to a skin temperature.

Accordingly the present invention provides an improved neuropathy device. In accordance with the embodiments described, the device provides the ability to take into account a reference temperature such as a reference skin or room temperature, so as to ensure that the thermal stimulus applied to the skin is appropriately corrected for ambient conditions, or a base skin temperature. This enables more reliable results to be obtained which are influenced less by the environmental factors. When a blood flow sensing arrangement is provided, more reliable results may be obtained, as blood flow is an autonomous factor, which is outside the control of the user. Thus the results are free from the subjective nature of conventional tests which involve asking the user to indicate when they can feel particular sensations.

The device of the present invention may be implemented as a portable hand-held device. The device advantageously includes a display e.g. for displaying results of testing to a user, and/or to enable a user to interact with the device. The device may also include a user interface to enable a user to for example input temperature settings or select temperature settings. The user interface may be provided as a touch screen or one or more buttons etc Rather than being in the form of a surface of a contact pad, the surface for applying a thermal stimulus may be provided as a surface of a plate, such as a foot plate. The device may include a suitable power source such as a battery, or may be mains operated, directly or indirectly e.g. through a computing apparatus. The device may be suitably connected to transmit results, e.g. over the Internet, or may be connected to a computing apparatus for so doing.

Although not shown, it will be appreciated that suitable limiters and other fail safe devices may be incorporated in the device to minimise risk to a patient in the event of misuse of the device or a fault.

The device may be implemented as a razor shaped device, particularly where the thermal stimulus is applied via a contact pad.

The area of skin which is tested may be any area useful for testing peripheral neuropathy. Typically the area may be an area of the skin of a foot, such as an area of the skin on the sole of a foot.

One exemplary embodiment of a device in accordance with the invention will now be described by reference to FIGS. 2-4. The device incorporates circuitry as illustrated by reference to FIG. 1.

The device 20 is in the form of a hand held, portable unit. The device has a housing which defines a handle portion 22 and a head portion 24. The handle portion 22 is configured to facilitate manual grasping by the user and includes operating buttons 26. The head portion includes a display screen 27 for displaying results of test and to enable the user to view settings etc. The head portion 24 also includes a thermal stimulus applicator surface 28 in the form of a pad surface. Adjacent the pad surface is an infrared transceiver 30. The receiver of the infrared transceiver 30 is operable to provide the temperature sensor for sensing a reference temperature, or alternatively, together with the transmitter of the transceiver is operable to sense blood flow. Although not shown, a set of one or more processors is housed in the handle portion 22 and a battery and the Peltier device and driver etc are located in the head portion.

In use the user holds the head of the device close to an area of skin to be tested and operates the detector of the infrared transceiver using the appropriate operating buttons to measure the temperature of the skin to provide a reference temperature. The device is then used to deliver a desired thermal stimulus taking into account the reference temperature by placing the applicator surface 28 against the skin and operating the device to deliver the stimulus. The device operates as described by reference to FIG. 1. The stimulus may be in accordance with a preprogrammed temperature setting which may be selected by the user in advance. The user may operate the transceiver 30 to determine a blood flow measurement indicative of the response to the stimulus. The result may be displayed on the screen 27. The device may be connected to a communications system to transmit results or to receive temperature settings etc.

1. A neuropathy test device comprising;
a thermal stimulus application arrangement for applying a thermal stimulus to a test area of skin in use,
wherein the neuropathy test device further comprises a temperature sensing arrangement for sensing a reference temperature for use in controlling the temperature of a thermal stimulus applied by the thermal stimulus application arrangement, wherein the reference temperature is a temperature of the ambient environment or a reference skin temperature.

2. The device of claim 1 wherein the thermal stimulus application arrangement comprises a thermal stimulus applicator surface for contacting the test area of skin to apply the thermal stimulus thereto, wherein the heat transfer arrangement is in thermal contact with the thermal stimulus applicator surface for delivering heat to and/or receiving heat from the thermal stimulus applicator surface.

3. The device of claim 2 wherein the thermal stimulus application arrangement comprises an applicator plate defining said thermal stimulus applicator surface, preferably wherein the applicator plate is a foot plate, or wherein the applicator surface is a surface of a pad.

4. The device of claim 2 further comprising a temperature sensing arrangement for sensing the temperature of the thermal stimulus applicator surface, preferably wherein the temperature sensing arrangement comprises a contact temperature sensor.

5. The device of claim 1 wherein the device further comprises a thermal stimulus temperature controlling arrangement operable to control a temperature of the thermal stimulus applied by the thermal stimulus application arrangement taking into account a reference temperature sensed by the temperature sensing arrangement.

6. The device of claim 5 wherein the device is arranged such that a sensed reference temperature is input to the temperature controlling arrangement for use by the temperature controlling arrangement in controlling the temperature of the applied thermal stimulus.

7. The device of claim 5 wherein the temperature controlling arrangement is operable to control the temperature of an applied thermal stimulus in accordance with one or more temperature settings taking into account the sensed reference temperature, preferably wherein the temperature settings are user selectable.

8. The device of claim 7 wherein the temperature controlling arrangement is in communication with a temperature setting arrangement for providing one or more temperature settings for the thermal stimulus to be applied.

9. The device of claim 7 wherein the temperature controlling arrangement is arranged such that the reference temperature is used to determine a base temperature level for use in controlling the temperature of the thermal stimulus in accordance with a given temperature setting.

10. The device of claim 4 wherein the temperature sensing arrangement for sensing the reference temperature comprises a non-contact, and preferably an infrared temperature sensor.

11. The device of claim 1 wherein the thermal stimulus application arrangement comprises a heat transfer arrangement.

12. The device of claim 11 wherein the heat transfer arrangement comprises a thermoelectric heat transfer device, preferably a Peltier device.

13. The device of claim 1 wherein the temperature sensing arrangement is operable to sense the reference temperature at least before the thermal stimulus application arrangement is operated.

14. The device of claim 1 wherein the thermal stimulus application arrangement is selectively operable to apply either a heat stimulus or a cold stimulus to the test area.

15. The device of claim 14 wherein the device is arranged to automatically select whether a hot or cold stimulus is applied, preferably wherein the arrangement is arranged to pseudo randomly select a temperature for an applied stimulus between given limits above and below the reference temperature.

16. The device of claim 1 wherein the device further comprises a blood flow sensing arrangement for sensing blood flow in the skin.

17. A neuropathy test device comprising;
a thermal stimulus application arrangement for applying a thermal stimulus to a test area of skin, and a blood flow sensing arrangement for sensing blood flow in the skin.

18. The device of claim 17 wherein the blood flow sensing arrangement comprises a transmitter for transmitting electromagnetic radiation and a detector for detecting electromagnetic radiation, preferably wherein the electromagnetic radiation is infrared radiation.

19. The device of claim 1 wherein the reference temperature sensing arrangement and/or the blood flow sensing arrangement is in communication with a set of one or more processors for controlling operation thereof and/or processing an output thereof and/or communicating the results of the sensing.

20. (canceled)

21. The device of claim 1, wherein the device is in the form of a hand held portable device.

22. (canceled)

23. A method of operating a device in accordance with claim 1 comprising the steps of operating the temperature sensing arrangement to obtain a reference temperature, and operating the thermal stimulus application arrangement to apply a thermal stimulus to the test area of skin taking into account the reference temperature.

24. A method of operating a neuropathy test device including a thermal stimulus application arrangement and a blood flow sensing arrangement comprising the steps of: operating the thermal stimulus application arrangement to apply a thermal stimulus to the test area of skin, and operating the blood flow sensing arrangement to determine the effect of the thermal stimulus upon blood flow.

25. (canceled)
